(19) United States
(12) Patent Application Publication
(10) Pub. No.: US 2025/0045561 A1
(43) Pub. Date: Feb. 6, 2025

(54) SYSTEM AND METHOD FOR INDUCTIVE LEARNING ON GRAPHS WITH KNOWLEDGE FROM LANGUAGE MODELS

(71) Applicant: NEC Laboratories Europe GmbH, Heidelberg (DE)

(72) Inventors: Filippo GRAZIOLI, Heidelberg (DE); Giulia CASSARA, Heidelberg (DE); Carolin LAWRENCE, Heidelberg (DE)

(21) Appl. No.: 18/704,945

(22) PCT Filed: Dec. 22, 2021
(86) PCT No.: PCT/EP2021/087326

§ 371 Date: Apr. 26, 2024

(30) Foreign Application Priority Data
Oct. 29, 2021 (EP) 21205661.8

(51) Int. Cl. G06N 3/042; G06N 5/022; G16C 20/50

(52) U.S. Cl. CPC G06N 3/042; G06N 5/022; G16C 20/50

(57) ABSTRACT

A computer-implemented method for inductive learning on graphs is provided. The graph includes a plurality of entities, where relationships exist between the plurality of entities, and where the plurality of entities and relationships have a name string. The method comprises creating for each entity of the plurality of entities of the graph a related text corpus, based on a respective name string of each entity. A pretrained language model is used to compute, from the related text corpus of each entity, a respective contextual entity embedding for each entity of the graph. A graph-based machine-learning (ML) model is trained, for each entity of the graph, the computed entity embeddings. These steps are repeated for unseen entities and the trained ML model is used to perform inductive predictions for the unseen entities.

### CROSS REFERENCE TO RELATED APPLICATIONS

This application is a U.S. National Phase application under 35 U.S.C. § 371 of International Application No. PCT/EP2021/087326, filed on Dec. 22, 2021, and claims benefit to European Patent Application No. EP 21205661.8, filed on Oct. 29, 2021. The International Application was published in English on May 4, 2023 as WO 2023/072421 A1 under PCT Article 21 (2).

### FIELD

The present invention relates to a system and a computer-implemented method for inductive learning on graphs, wherein the graph includes a plurality of entities, wherein relationships exist between the entities, and wherein the entities and relationships have a name string.

### BACKGROUND

In the context of graph-based machine learning (ML), inductive learning consists in training a model on a set of known nodes/entities (and relationship types) and to test the obtained model on unseen nodes/entities (and relationship types). Supporting the inductive learning setting is crucial for a graph-based ML system, as this allows unseen entities to be frequently introduced in the graph without compromising the functionality of the system and without the need of frequent re-training steps.

Without lack of generality, it is possible to divide every graph-based ML problem into the following ontological categories: (1) nodes with numerical/categorical features, and (2) nodes without numerical/categorical features.

When graphs present nodes with numerical/categorical features (as exemplarily shown in FIG. 1), inductive learning does not present major issues. A broad body of literature presents solutions for this kind of graph-based ML problems, for references see, e.g., W. L. Hamilton, R. Ying, J. Leskovec: “Inductive Representation Learning on Large Graphs”, 31^{st} Conference on Neural Information Processing Systems (NIPS 2017), Long Beach, CA, USA.

On the other hand, when nodes do not present numerical/categorical features (as exemplarily shown in FIG. 2), inductive learning turns into a challenging problem. Various methods have been recently proposed for the solution of graph-based inductive learning tasks. The most trivial approach is to limit the problem to the transductive learning setting. In this case, even though nodes do not have features, all entities have an ID, which is then encoded. During training, a transformation of the embedding to a hidden embedding space is learned. This leads to the main limitation of the transductive setting: the model cannot work with unseen entities, as the ID embedding is unknown.

A possible way to tackle the inductive learning problem is by leveraging on the topology of the graph to compute an embedding of the nodes. The double radius vertex scheme proposed by K. K. Teru, E. Denis, W. L. Hamilton: “Inductive Relation Prediction by Subgraph Reasoning”, ICML 2020, arXiv: 1911.06962 is an example of this kind of approach. The main limitation of this is that unseen entities might come with only partial connections to the rest of the graph. Indeed, the goal of link prediction is also to discover missing connections. Leveraging only on the topology is therefore prone to computing biased embeddings, when many connections are missing at test time.

B. Wang et al.: “Inductive Learning on Commonsense Knowledge Graph Completion”, in 2021 International Joint Conference on Neural Networks (IJCNN), Shenzhen, China 18-22-7-2021, arXiv: 2009.09263v2 2021 also consider the inductive case for the task of knowledge graph completion. Specifically, the proposed method utilizes a language model (or word embedding model) to create a textual representation of a new, previously unseen entity. However, their architecture requires to learn embeddings implicitly which generally results in an inferior textual representation.

K. V. Ramanan et al.: “A Joint Training Framework for Open-World Knowledge Graph Embeddings”, in Conference Paper, Automated Knowledge Base Construction (AKBC) 221-6-2021 also consider the inductive setting and propose an architecture that combines language models (LMs) and Commonsense knowledge graphs (KGs). The architecture jointly learns embeddings for KG entities from descriptions and KG structure for open-world knowledge graph completion by aligning the two spaces. However, this approach approves to be a disadvantageous in terms of efficiency since it requires training on the descriptions base.

### SUMMARY

In an embodiment, the present disclosure provides a computer-implemented method for inductive learning on graphs, wherein a graph includes a plurality of entities, wherein relationships exist between the plurality of entities, and wherein the plurality of entities and relationships have a name string, the method comprising: (a) creating for each entity of the plurality of entities of the graph a related text corpus, based on a respective name string of each entity; (b) using a pretrained language model to compute, from the related text corpus of each entity, a respective contextual entity embedding for each entity of the graph; training a graph-based machine-learning (ML) model by using, for each entity of the graph, the computed entity embeddings; and repeating, for unseen entities, steps (a) and (b) and using the trained ML model to perform inductive predictions for the unseen entities.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 is a schematic view illustrating a graph according to prior art where nodes have numerical/categorical features;

FIG. 2 is a schematic view illustrating a graph according to prior art where nodes have no numerical/categorical features;

FIG. 3 is a schematic view illustrating the architecture of a system for inductive learning on graphs with knowledge from language models in accordance with an embodiment of the present invention;

FIG. 4 is a schematic view illustrating a working principle of a neighbor collector implemented in a system for inductive learning according to an embodiment of the present invention;

FIG. 5 is a schematic view illustrating a working principle of a corpus collector implemented in a system for inductive learning according to an embodiment of the present invention;

FIG. 6 is a schematic view illustrating a process of creating an entity embedding according to an embodiment of the present invention when the entity name is present in the extracted sentences; and

FIG. 7 is a schematic view illustrating a process of creating an entity embedding according to an embodiment of the present invention when the entity name is not present in the extracted sentences.

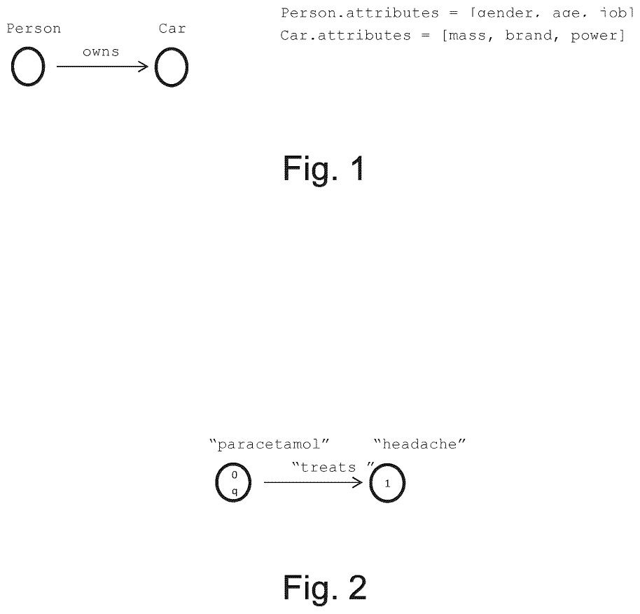

### DETAILED DESCRIPTION

In accordance with an embodiment, the present invention improves and further develops a method and a system of the initially described type in such a way that graph-based inductive learning is enabled under the following edge boundary conditions, which often occur in real-world applications: (1) nodes and relationships may have no numerical/categorical features; (2) unseen test nodes might lack connection with the train graph, or even be disconnected; and/or (3) nodes and relationships (only) have a name string.

In accordance with another embodiment, the present invention provides a computer-implemented method for inductive learning on graphs, wherein the graph includes a plurality of entities, wherein relationships exist between the entities, and wherein the entities and relationships have a name string, the method comprising: (a) creating for each entity of the graph, based on the entity's name string, a related text corpus; (b) using a pretrained language model to compute, from the related text corpus of an entity, a respective contextual entity embedding for each entity of the graph; training a graph-based machine-learning, ML, model by using, for each entity of the graph, the computed entity embeddings; and repeating, for unseen entities, steps (a) and (b) and using the trained ML model to perform inductive predictions for the unseen entities.

Furthermore, in accordance with another embodiment, the present invention provides a system for inductive learning on graphs, wherein the graph includes a plurality of entities, wherein relationships exist between the entities, and wherein the entities and relationships have a name string, the system comprising one or more processes that, alone or in combination, are configured to provide for the execution of the following steps: (a) creating for each entity of the graph, based on the entity's name string, a related text corpus; (b) using a pretrained language model to compute, from the related text corpus of an entity, a respective contextual entity embedding for each entity of the graph; training a graph-based machine-learning, ML, model by using, for each entity of the graph, the computed entity embeddings; and repeating, for unseen entities, steps (a) and (b) and using the trained ML model to perform inductive predictions for the unseen entities.

Embodiments of the present invention provide a computer-implemented method and system to operate inductive machine learning on graphs by leveraging on knowledge from language models (LMs). With the goal to allow maximum generality, the nodes in the graph only need to present a name string for the invention to be applicable. For each node in the graph, a representation (embedding) is computed, for instance by (1) mining related text and embedding it with LMs, and/or (2) creating for each node a neighbor textual corpus and using LMs to create contextual embeddings. The system and method according to embodiments of the invention support the following tasks: link prediction, graph classification, node classification, as well as general representation learning. Both Graph Neural Networks (GNNs) and Knowledge Graph Embeddings (KGE) methods are supported.

The present invention generally relates to a system and method for inductive learning on graphs with knowledge from language models. Initially, text related to an entity of a graph may be extracted with the help of some other database or by mining text form internet. Once the text is extracted, the relationship between the node/entity and its neighbor may be extracted and converted to natural language (only applicable if a node has neighbors). The system further may be integrated with language models, for example BERT, etc. that may be used to create contextualized token for each sentence of the entity related text. Once tokens are created, sentence embeddings may be computed, e.g. by averaging the token embeddings. Further, with the help of sentence embeddings, entity embeddings may be computed. In the same way, new/unseen entity embeddings may be computed by the system. To predict new relations between new/unseen entities and old entities, k-nearest and k+1 nearest neighbors may be identified by computing a distance metric between the new entities' language model embedding and the language model embedding of all other entities and aggregated to get a combined score.

According to embodiments of the invention, the language model may be used to compute a representation for the entities, which are in the initial graph. These representations may serve as numerical feature vectors that may be assigned to the respective entities. A graph model, for instance a graph neural network (GNN) or knowledge graph embedding (KGE) method, may utilize the numerical feature vectors to get trained and to operate inductively.

According to embodiments of the invention it may provided that predictions obtained for a given entity are combined with those obtained using its k-nearest neighbors. This allows to compute a joint (and highly robust) score. Moreover, identifying the k-nearest neighbors of new test entities allows to augment the graph with new triples in a self-supervised fashion, which can then be used by the graph model for learning at the next training iteration.

According to an embodiment of the invention, for each entity in the graph a related textual corpus is embedded with a pre-trained LM (whose weights are frozen) and aggregated in order to compute a single entity embedding. In case an entity has neighbor entities within the graph, a neighbor corpus may be extracted by considering all relationships that compose a neighbor and converting them into natural text. All sentences may then be embedded and the entity contextual embeddings may be aggregated to form a single representation. This allows to generate node features for unseen entities and enables the graph model (e.g. a GNN or a KGE model) to perform inductive predictions for unseen entities.

Using a textual corpus for computing the single entity embeddings makes it possible to learn a contextualized embedding for an entity. In contrast to a non-contextualized embedding, this leads to better results, since the graph model, trained on the basis of the contextualized embeddings, is able to understand what the embeddings mean, such that the model can be used in an inductive setting.

According to an embodiment of the invention, when the model is used to operate predictions for a new entity that has not been seen during training, its k-nearest neighbors may be identified by computing a distance metric between the new entity's LM embedding and the LM embeddings of all other entities. Using the nearest entities in the LM embedding space to run predictions for the graph model enriches the graph model with information from the LM space. Predictions may be operated for all k+1 entities and then aggregated to obtain a combined scored.

According to embodiment of the invention, based on the aggregated predictions from the k+1 entities, a set of reliable predictions may be determined, e.g. via a threshold. These predictions may then be explicitly added to the knowledge graph, the model may be updated and predictions may be rerun.

In contrast to the inductive learning approach described in the prior art document mentioned in the introduction (B. Wang et al.: “Inductive Learning on Commonsense Knowledge Graph Completion”), where only the name of the new entity itself is embedded, embodiments of the invention propose to embed entire sentences in which the entity occurs. In the case of embedding just the entity, the language model has to rely on having to see this particular word before in a meaningful context during the training of the language model. This means the embedding must be learned implicitly. In contrast, embodiments of the invention enable to supply exactly the sentence which is known to be relevant for the entity in the current usage context, and this can be used to explicitly encode relevant information. This explicit embedding is more powerful and can therefore yield a better textual representation.

In contrast to the inductive learning approach described in the further prior art document mentioned in the introduction (K. V. Ramanan et al.: “A Joint Training Framework for Open-World Knowledge Graph Embeddings”), where embeddings for KG entities are jointly learned from descriptions and KG structure for open-world knowledge graph completion by aligning the two spaces, the system according to embodiments of the invention does not train on the description space. This means that the system does not fine-tune the language model, which results in an increased efficiency. Furthermore, the framework according to embodiments of the invention is agnostic to the pre-trained language model and, most important, according to embodiments of the invention the description embedding is simply “transferred” in the space of the knowledge graph by initializing and learning the embeddings using a graph-based ML model.

Embodiments of the present invention can be suitably applied in different technological fields. Use cases span various sectors such as finance, biomedical and public safety. Advantageously, the present invention would allow to also operate in a setting where an entity has not been seen during training. This is for example a known shortcoming of KBlrn (for reference, see A. Garcia-Duran, M. Niepert: “KBLRN: End-to-End Learning of Knowledge Base Representations with Latent, Relational, and Numerical Features, arXiv: 1709.04676).

There are several ways how to design and further develop the teaching of the present invention in an advantageous way. To this end it is to be referred to the dependent claims on the one hand and to the following explanation of preferred embodiments of the invention by way of example, illustrated by the figure on the other hand. In connection with the explanation of the preferred embodiments of the invention by the aid of the figure, generally preferred embodiments and further developments of the teaching will be explained.

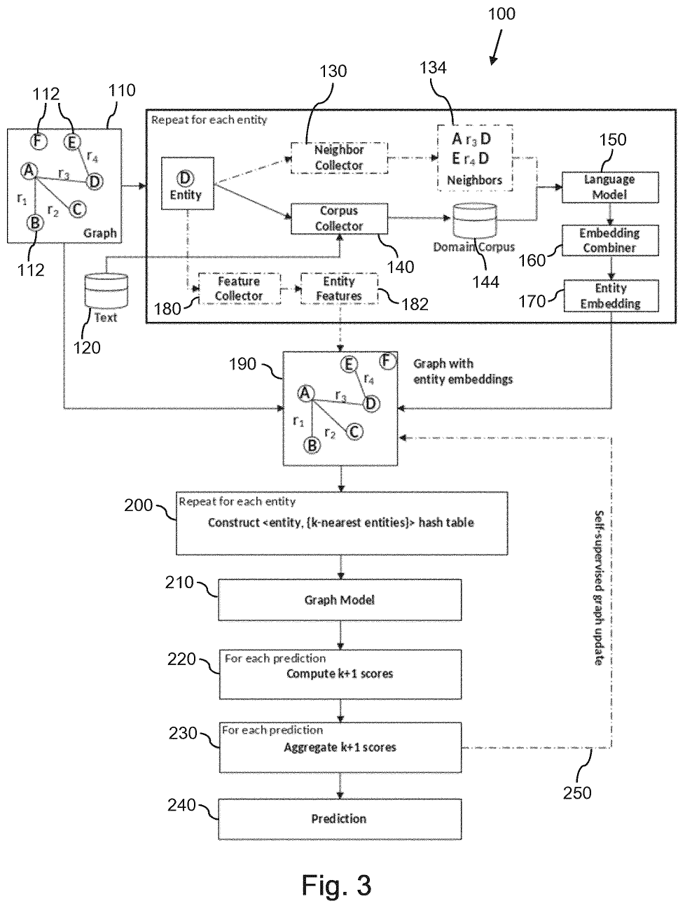

Embodiments of the present invention relate to a system and method for inductive learning on graphs with knowledge from language models (LMs). FIG. 3 illustrates an exemplary system architecture 100 according to an embodiment of the present invention. Generally, it should be noted that the following description of illustrated embodiment is carried out in a prediction-agnostic fashion, as the concrete prediction of the graph-based model does not place any constraints on the system architecture 100. Furthermore, since the system 100 can implement various graph-based ML algorithms to operate, the following description does not place emphasis on a specific machine learning approach. In the following embodiment descriptions, without lack of generality, multiple concrete implementations will be described, together with various algorithmic approaches and use cases.

Embodiments of the present invention rely on an adoption of LMs for the computation of numerical feature vectors for graph nodes. This allows to generate representations based on a simple name string and, therefore, to enable inductive learning when (1) no node feature vectors are otherwise available (although, of course, they could be), (2) new unseen test entities are not (or only partially) connected to the rest of the graph, and/or (3) the only node attribute is its name.

FIG. 3 depicts an embodiment of the system 100, wherein in particular the dashed line arrows and boxes illustrate optional steps and components. In the following, each module of this system 100 will be described in detail.

### Graph

The graph 110 constitutes the main input of the system 100. As shown in FIG. 3, A, . . . , F denote the nodes 112 of the graph 110 (equivalently referred to as entities in the present disclosure). r1, . . . , r4 denote existing relationships between the nodes 112.

For the system 100 to work, the only requirement that may be placed on the graph 110 is that all nodes/entities 112 have a string name (herein sometimes also referred to as name attribute). Relationships must also have a string, which expresses the relationship type. The nodes 112 in the graph 110 are allowed to change over time. The system 100 allows inductive learning, i.e. predictions can be made for entities 112 which are not present at training time.

### Text

The text input consists of a database of textual data 120. The database 120 is configured in such a way that it includes, for each entity 112 of the graph 110, a related body of text. The textual data 120 contained in the database can have any source: for instance, it may come from domain-specific scientific literature, news, Wikipedia, book chapters, etc. Text could also be mined from the Internet on the fly, and must not necessarily be stored on a disk. It is to be understood that the present invention is in no way limited as what regards the nature of the text database 120.

### Neighbor Collector

The neighbor collector 130 may be used for those nodes 112 that have at least one neighbor within the graph 110. Since the graph 110 might comprised nodes without any neighbors, the neighbor collector 130 can be regarded as an optional component of the system 100. According to an embodiment, the neighbor collector 130 can be triggered on and off, based on whether or not a neighboring node is available for a given node 112.

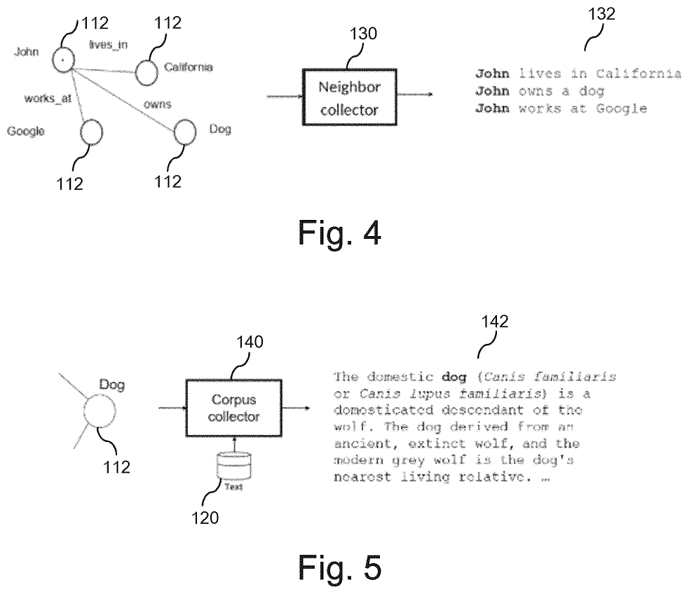

According to embodiments, the neighbor collector 130 may be configured to collect, given a node 112, all neighboring nodes and extract sentences from the identified set of relationships. Rule-based processing of such sentences may also be performed, if applicable, as an optional step, so that a triplet (expressed as, e.g., (h, r, t), meaning that the two nodes/entities h and t are linked through relation r) can be expressed as natural language, as described in some more detail in connection with FIG. 4. In this context, it should be noted that, following the same principle, the system 100 can support k-hops neighbors (not only 1-hop neighbors, as displayed in FIG. 4).

As exemplarily shown in FIG. 4, a node 112 with name attribute ‘John’ is assumed to have three neighboring nodes 112 within the graph 110, namely the nodes with the name attributes ‘California’, ‘Dog’, and ‘Google’. The relationships between the node ‘John’ and these neighboring nodes have the name attributes ‘lives_in’, ‘owns’ and ‘works_at’. The neighbor collector 130 collects all neighboring nodes and extracts sentences from the identified set of relationships, which are then expressed as natural language as ‘John lives in California’, ‘John owns a dog’ and ‘John works at Google’.

The triplets 132 expressed as natural language may then be collected in a neighbor corpus 134, as shown in FIG. 3.

### Corpus Collector

FIG. 5 schematically illustrates the working principle of a corpus collector 140 according to embodiments of the present invention. Given an entity 112 (and its name string), the corpus collector 140 may be configured to query the text database 120 to extract a related text corpus. The extracted text corpus 142 shall be either (1) about the entity or (2) written by the entity (see also embodiments below). Although in the scenario shown in FIG. 4, the entity name, i.e. the name string of the respective entity 112 (‘dog’ in the illustrated case) explicitly occurs in the extracted text corpus 142, this need not necessarily be the case.

The corpus collector 140 may be successively applied to each node 112 of the graph 110.

The entirety of the extracted text corpuses 142 may then be collected in a domain corpus database 144, as shown in FIG. 3.

### Language Model

The system 100 further comprises an LM 150 that is configured to compute, based on the content of the domain corpus 144, a representation (embedding) for each node 112 of the graph 110. Optionally, the neighbor corpus 134—if available—may also be embedded.

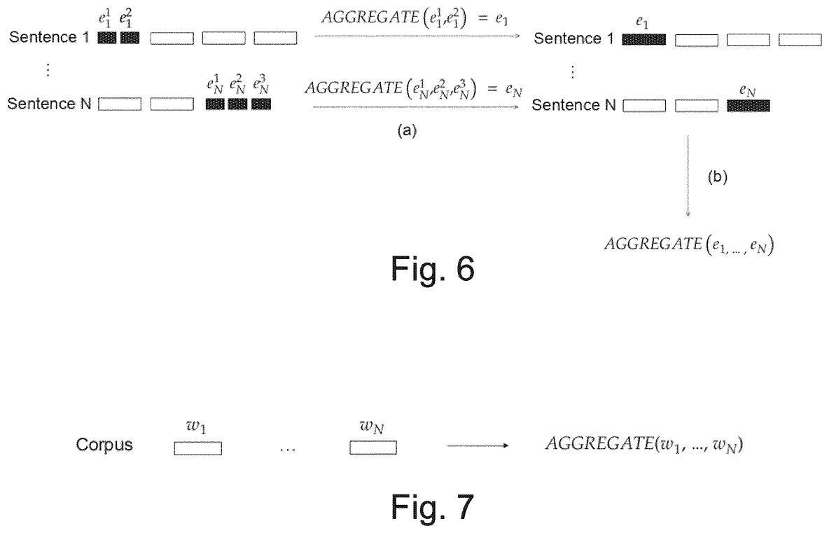

Doing so will lead to having multiple embedded sentences, as schematically shown in FIG. 6, where each word of the sentences contained in the domain corpus 144 and possibly the neighbor corpus 134 is contextually embedded. Contextual embedding of a word means that its representation changes based on the context and the words that appear in the sentence. Specifically, it may be provided that the LM 150 generates embedded sentences, in which each word is represented as a numeric vector. The LM 150 is not updated by the system 100 and its weights are frozen. According to an embodiment of the invention, the LM 150 may be implemented by using a state-of-the-art transformer-based LM, which are generally trained in a self-supervised fashion on huge text corpora. They can directly be used to create token embeddings and, based on this, sentence embeddings can be computed. For instance, a model like BERT (for reference, see J. Devlin et al.: “BERT: Pre-training of Deep Bidirectional Transformers for Language Understanding”, 2019, arXiv: 1810.04805, the content of which is incorporated herein by way of reference) may be used to implement LM 150.

### Embedding Combiner and Entity Embedding

As explained above, the LM 150 may provide embedded sentences, where each word is represented as a numeric vector. Given a certain node/entity 112, depending on whether or not its name string is present in the sentences (collected in the domain corpus 144 and possibly in the neighbor corpus 134), various approaches are possible for the creation of the final entity embedding. Some possible techniques that may be executed by embedding combiner component 160 and entity embedding component 170 are presented below.

If the node name is included in the extracted sentences 132, 142, an embedding of the entity name will be available for each extracted sentence, as exemplary shown in FIG. 6. Here, each rectangle represents the embedding of a word in a given sentence computed by the LM 150. The dark rectangles refer to the contextual embeddings of the considered node name.

A possibility for creating the final embedding is to aggregate the embeddings of all extracted sentences 132, 142. The AGGREGATE ( ) function of FIG. 6 depicts an abstract procedure for aggregating entity embedding. Concrete possible instantiations are, e.g., the mean, the sum, the max-pooling, or the average-pooling. The invention is in no way limited in this regard.

As depicted in FIG. 6, the AGGREGATE ( ) function may operate on two levels. First, an intra-sentence aggregation is needed to combine the embeddings of entities which present more than one token. This is shown in variant (a), wherein e represents a token of a respective entity string, such that eᵢ^{j} denotes token embedding j in sentence i. Second, once all entities are represented as a single embedding in each sentence (denoted eᵢ for sentence i), an inter-sentence aggregation shall take place (as shown in variant (b)), to combine entity embeddings eᵢ across different sentences so that a single representation for the entity, denoted AGGREGATE (e₁, . . . , e_{N}), is computed.

The approach described above is not possible when the text corpuses 134, 144 do not include the name of the respective entity 112. An example of this is e.g. electronic health records written by physicians for a given patient. The patient node in the graph is normally named with an identifier, but this name string is most of the time not available in the electronic health records.

FIG. 7 depicts a possible way to compute entity embeddings from text when the corpus 134, 144 consists of sentences, which concern the respective entity 112 without including a string with the entity name (i.e. sentences do not include any entity string). Each rectangle is a word embedding computed with the LM 150. As such, in contrast to FIG. 6, wᵢ denote representations (embeddings) of whole sentences. The AGGREGATE( ) function, like in the previous example, aims at combining all word embeddings in a single representation. For this purpose, mean, sum, average-pooling are some exemplary viable alternatives. Of course, this approach may also be used even if the entity name is present.

### Feature Collector and Entity Features

Although the present invention mainly aims at solving the inductive learning problems, which are particularly challenging when the nodes 112 do not have numerical/categorical features, embodiments of the system 100 may nonetheless support a setting in which nodes 112 do have feature vectors. To this end, the system 100 may include a feature collector 180 that is configured to get the original node features 182, which are then going to be combined with those computed by the LM 150 and used by the graph model 210 for training and prediction, as will be described in detail further below. The entities features 182 can be combined with those extracted by the LM 150 e.g. with a simple concatenation step, but also with more sophisticated techniques.

According to an embodiment, the feature collector 180 can be triggered on and off, based on whether or not entity features 182 are available for a given node 112.

Graph with Entity Embeddings

Entity embeddings computed with the steps described above are then associated to the respective entities 112 in order to construct a graph 190 whose vertexes have numerical embeddings as attributes. If numerical/categorical entity features 182 are provided by the feature collector 180, they can be combined with the entity embeddings, as described above, e.g. simply be concatenating them to the entity embeddings.

### Construct <Entity, {K-Nearest Entities}>Hash Tables

At this point, each entity 112 has a numeric representation which in the present disclosure will be referred to as embedding. As shown at 200, for each entity 112, its k-nearest entities are identified. The concept of “nearness” can be embodied by any metric which computes a distance in a vector space of the same dimensionality of the embeddings. The Euclidean distance is an option. Alternatives include, but not limited to, the cosine similarity, the geometric distance or the L1 norm. Once, for all entities 112, the k-nearest entities are identified, a mapping between an entity 112 and its k-nearest entities may be constructed, e.g. as a hash table (or any other mapping) in the form <entity, {k-nearest entities}>.

### Graph Model

According to embodiments of the present invention, the system 100 is configured to generate a graph model 210, based on the entity embeddings and the k-nearest neighbor identification, as described above. For the graph model 210, the following two options are possible.

According to one embodiment, the graph model 210 may be constructed by combining Knowledge Base Completion (KBC) techniques with Knowledge Graph Embedding (KGE) models. In the case of KBC, the graph model construction is based on a set of entities and a set of relations. A graph in this context is described by a set of triples. A triple consists of a head, relation and tail. Head and tail are entities, which are the nodes of the graph. Relations are edges that hold between nodes. The goal of KBC is to predict new relations (edges) between entities in the knowledge graph. The best models for this are KGE models, such as DistMult (for reference, see B. Yang et al.: “Embedding Entities and Relations for Learning and Inference in Knowledge Base”, 2015, https://arxiv.org/abs/1412.6575). However, they only work if an entity has been seen during training time.

In contrast, the system 100 according to embodiments of the present invention allows to add a new entity at test time (i.e. without the entity being present during training time), since it is not required that any relation exists for this entity yet. According to embodiments of the invention, the new entity can be encoded using the language model 150 and the resulting embedding can directly be used by the KGE model. The KGE can then predict to which other entities the new entity should connect via which relation. Optionally, one can add one more learnt transformation to the training of the KGE model which transforms the embedding from the language model to the embedding then used by the KGE model.

According to another embodiment, the graph model 210 may be constructed by means of Graph Classification or Node Classification with Graph Neural Networks (GNN). Given the graph 190 with entity embeddings, a GNN can be trained and used to either classify the graph 190 or to classify nodes 112 in the graph 190. To describe nodes 112 in the graph 190 there are two options. For the first option, each node 112 is described by a set of features. New nodes can be added at test time as long as the new node can be described with the same set of features. For the second option, each node 112 is described by a unique name. Generally, in this case, new nodes cannot easily be added at test time. However, the system 100 according to embodiments of the present invention addresses this scenario, as, in contrast to prior art solutions, with the present invention it is now possible to add new nodes. To this end, it may be provided that a new node is encoded using the language model 150 (as described above), wherein the resulting embedding can directly be used by the GNN.

### Compute K+1 Scores

According to embodiments of the present invention it may be provided that, for any type of prediction (e.g. link prediction, node classification), the graph model 210 described above not only computes a single set of predictions or scores, but a set of k+1 predictions or scores, as indicated at 220 in FIG. 3. This may be achieved by using the <entity, {k-nearest entities}>hash tables, previously constructed (cf. step 200 in FIG. 3) for all entities 112. Iteratively, the graph model 210 may consider the entity embedding, as well as the embeddings of its k-nearest entities. This way, a set of k+1 predictions is obtained each time the graph model 210 performs a prediction.

As an example, the 3-tuple (h, r, t) is defined as a triple. (h, r, t) represents a directed edge of type r from entity h (the “head”) to t (the “tail”). It is assumed that link prediction shall be operated in the form (entity, r,?), where entity is a test entity. In this scenario, the models may learn a function f: R^{embedding_dim}×R^{embedding_dim}→R^{num_of_nodes}, such that f(h, r) estimates a probability distribution over tail nodes. According to an embodiment of the invention, ∀hϵ{k−nearest entities}∪{entity}, a f(h, r) score is computed.

### Aggregate K+1 Scores

For each prediction operated by the graph model, the previously described step (as shown at 220 in FIG. 3) has provided k+1 predictions. In a next step, as shown at 230, these predictions may be aggregated. Simple approaches for doing this include, but not limited to, e.g. a majority vote or the mean of the k+1 predictions. Alternatively, the k+1 predictions can also be aggregated by means of a learnable block, such as self-attention.

### Prediction

The aggregated prediction computed at the previous step 230 can be provided as the prediction output 240 of the prediction system 100.

### Self-Supervised Graph Update

According to an embodiment of the present invention, the prediction system 100 may incorporate a mechanism for self-supervised creation of new training triples, implemented e.g. in form of a feedback loop 250. In this context, it may be provided that each time a prediction is performed for a new test entity, k-nearest entities are identified, as described above for step 200. Subsequently, k+1 predictions may be performed and aggregated as described above for step 220 and 230, respectively.

As an example, the test triple (entity, r, t) is considered, where entity is a new test entity, not observed during training. ∀hϵ{k−nearest entities}, k new triples (h, r, t) can be constructed and added to the training set. At the next training iteration, the graph model 210 has then access to these new triples.

According to embodiments of the invention, all steps from the step of computing the k+1 scores (step 220) to the step of the self-supervised graph update (step 250) are repeated N times, where N may be either a user-defined hyperparameter or the number of steps that may be necessary for a test metric to achieve convergence.

It should be noted that although the idea of triple, i.e. (h, r, t), is mainly adopted in the KGE literature, the reasoning presented in the present disclosure holds for GNNs too. A triple is in fact a directed edge in a graph.

According to an embodiment that combines the components explained above in connection with FIG. 3, the present invention provides a method for inductive learning on graphs with transfer learning from language models, the method comprising the steps of
1) given a node and its name string, get a related textual corpus either by querying a database, or mining text from the Internet (e.g. as described in the above section ‘Text’);
2) given a node and its neighbor, extract the available relationships and convert them into natural language with a rule-based approach (optional, only applicable if a node has neighbors) (e.g. as described in the above section ‘Neighbor collector’);
3) using a LM extract the entity embeddings from the natural language obtained from steps (1) and (2) (e.g. as described in the above sections ‘Language model’, ‘Corpus collector’, ‘Embedding combiner and entity embedding’) including
   a) computing for each sentence the contextualized token embeddings (e.g. using BERT);
   b) using token embeddings to compute a sentence embedding (e.g. by averaging the token embeddings); and
   c) combining each sentence embedding into a final entity embedding (e.g. by averaging the sentence embeddings);
4) training a graph-based ML model using, for each entity, the embeddings obtained from the LM (e.g. as described in the above section ‘Graph model’);
5) for unseen entities, repeating steps (1), (2), (3) and using the graph model from step (4) for inductive predictions;
6) computing a distance metric between the entity embeddings, in order to identify the k−nearest neighbors set for each entity (e.g. as described in the above section ‘Construct <entity, {k-nearest entities}>hash tables’);
7) for new entities, at test time, combining the k+1 scores obtained by also considering the k-nearest neighbors (e.g. as described in the above sections ‘Compute k+1 scores’ and ‘Aggregate k+1 scores’);
8) updating the graph using triples obtained substituting a test entity with its k-nearest neighbors (e.g. as described in the above section ‘Self-supervised graph update’); and
9) repeating step 6 through 8 until a desired/predefined end condition is reached.

### Technical Use Case: Biomedical AI for Drug Re-Purposing/Discovery

Graph-based ML systems for drug repurposing and drug discovery leverage graphs that often present genes, proteins, drugs, adverse effects, diseases, etc. Most of the time, knowledge graphs are built or enriched by text mining a large-scale literature repository. In this situation, entities have no numerical features but IDs. In case a new gene/protein is discovered, for instance, it may usually be required to manually add the entity to the Knowledge Graph and retrain the old Knowledge Graph model. However, there are situations in which retraining is impossible because it is time-consuming, and the novel entity has no prior relations with the existing knowledge graph. Nevertheless, it may be necessary to predict something about this new entity. Therefore, these systems could benefit from the inductive learning method proposed in the present disclosure when new entities are only available at test time, and when they have only a text associated with them. A drug discovery system would then allow to (inductively) discover new chemicals that can cure a given disease and would be embedded in an apparatus capable of optimizing and eventually creating the drug. In the following, for this specific embodiment, all the components of the system will be described, thereby making reference to FIG. 3.

In case of a drug discovery system, the graph 110 could be a network that represents as entities 112 genes, proteins, drugs, adverse effects, diseases, and the like. Relationship types established between the entities 112 of the graph 110 could include, but not limited to:
drug-treats-disease
drug-interacts with-drug
gene-is associated with-disease
drug-influences expression of-gene
gene-regulates-gene
disease-is similar to-disease
drug-can cause-a side effect

The textual data contained in the database 120 may include, in particular, scientific/medical literature related to the entities (e.g., related to the genes, drugs, diseases, etc. in the network).

Based on this information, the drug discovery system may operate in the same way as described above for the prediction system 100 of FIG. 3. Specifically, in the case of a drug discovery system, a prediction 240 may include the discovery of new “drug-treats-disease” relationships not presented in the initial graph 110. A new “drug-treats-disease” relationships may be considered as “drug repurposing”, i.e. finding new targets not yet known for a specific drug.

The drug discovery system may be configured to execute an inductive step, in which a new set of genes/proteins related to a disease is added to the graph 190 after a KGE/GNN is trained. Predictions may then be performed by taking this new set of entities into account. This allows predicting whether a drug in the network can treat any of the new diseases (not seen at training time).

### Technical Use Case: Biomedical AI for Patient Treatment

Patients can be linked to similar other patients by grouping individuals based on a set of medical/physiological features. This allows to create a graph 110 with patients as entities 112 and links between patients indicating similarity. At the same time, for patients consistent bodies of text are often available (e.g. Health Records written by physicians). If a new patient is added to the graph 110, embodiments of the present invention allow to create representations for the patients starting from their textual Health Records and allows graph models 210 to operate inductively on new patients which were not available at training time. Based on this, a GNN can for example predict the dosage of a medication that should be given to a patient. This suggestion can then either be administered by staff or medical devices or can automatically be adjusted.

### Technical Use Case: Public Safety/Law Enforcement

Given a network of people, in accordance with an embodiment, the present invention detects if a person or group of people is in danger of being radicalized. According to an embodiment of the invention, if a new person joins the network, at first an appropriate representation for this new person will be computed. This can be done by using text written by the new person as the domain corpus 144 and then the representation may be computed based on the written text. Based on the predicted danger of radicalization, a monitoring system can be adjusted with regards to what is recorded about the person or the group. For example, if a group is deemed in danger of being radicalized, drones could more often patrol the area the group operates in.

As will be appreciated by those skilled in the art, apart from the use cases explicitly described above, implementation of embodiments of the present invention is straightforward for a variety of further applications.

Many modifications and other embodiments of the invention set forth herein will come to mind to the one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1. A computer-implemented method for inductive learning on graphs, wherein a graph includes a plurality of entities, wherein relationships exist between the plurality of entities, and wherein the plurality of entities and relationships have a name string, the method comprising:
(a) creating for each entity of the plurality of entities of the graph, a related text corpus, based on a respective name string of each entity;
(b) using a pretrained language model (150) to compute, from the related text corpus of each entity, a respective contextual entity embedding for each entity of the graph;
training a graph-based machine-learning (ML) model by using, for each entity of the graph, the computed entity embeddings; and
repeating, for unseen entities, steps (a) and (b) and using the trained ML model to perform inductive predictions for the unseen entities.

2. The method according to claim 1, further comprising:
computing a distance metric between the entity embeddings and identifying, based on the distance metric, k-nearest neighbors set for each entity of the plurality of entities of the graph; and
computing, by using the trained ML model, a set of k+1 predictions for each entity including a prediction for the respective entity itself and k predictions for the k-nearest neighbors.

3. The method according to claim 2, further comprising:
aggregating the computed k+1 predictions to obtain an aggregated prediction for each entity; and
providing the aggregated predictions as a prediction output.

4. The method according to claim 3, further comprising:
updating the graph using triples obtained by substituting a test entity with k-nearest neighbors of the test entity.

5. The method according to claim 2, further comprising:
repeating the steps of claim 2 until a desired or configurable end condition is reached.

6. The method according to claim 1, wherein the related text corpus for each entity of the plurality of entities of the graph is created by querying a database of textual data and/or by mining text from an external source.

7. The method according to claim 1, further comprising:
extracting, for each entity of the plurality of entities of the graph and its neighbor, available relationships and converting the available relationships into natural language with a rule-based approach; and
using, by the pretrained language model, the converted natural language to compute the contextual entity embedding for the respective entity of the graph.

8. The method according to claim 1, wherein computing the contextual entity embeddings for the plurality of entities of the graph comprises:
computing, for each sentence of the related text corpus created for each entity and/or the natural language extracted from relationships available for each entity, contextualized token embeddings;
using the computed contextualized token embeddings to compute a sentence embedding; and
combining each sentence embedding into a final entity embedding.

9. The method according to claim 8, wherein the final entity embedding is created by aggregating the sentence embeddings by means of an aggregation function, wherein the aggregation function operates to calculate the mean, the sum, the max-pooling, or the average-pooling.

10. The method according to claim 1, wherein the graph-based ML model is implemented based on a Knowledge Base Completion (KBC) with Knowledge Graph Embedding (KGE) model.

11. The method according to claim 1, wherein the graph-based ML model is implemented based on Graph Classification or Node Classification with Graph Neural Networks (GNN).

12. The method according to claim 1, wherein the entities of the graph represent biomedical entities including drugs, genes, proteins and diseases,
wherein the inductive learning is used as part of a drug discovery system to optimize and create a drug,
wherein the graph represents biomedical relationship types between the plurality of entities including relationships of at least one of the types ‘drug-treats-disease’, ‘drug-interacts with-drug’, ‘gene-is associated with-disease’, ‘drug-influences expression of-gene’, ‘gene-regulates-gene’ and ‘disease-is similar to-disease’, and
wherein the related text corpus for each entity is extracted from scientific/medical literature related to the plurality of entities.

13. The method according to claim 12, wherein the predictions include predictions whether a drug in the graph can treat a particular disease related to a new set of genes/proteins that is added to the graph after the ML model has been trained.

14. A system for inductive learning on graphs, in particular for execution of a method according to claim 1, wherein a graph includes a plurality of entities, wherein relationships exist between the plurality of entities, and wherein the plurality of entities and relationships have a name string, the system comprising one or more processes that, alone or in combination, are configured to provide for the execution of the following steps:
(a) creating for each entity of the plurality of entities of the graph a related text corpus, based on a respective name string of each entity;
(b) using a pretrained language model to compute, from the related text corpus of each entity, a respective contextual entity embedding for each entity of the graph;
training a graph-based machine-learning, ML, model by using, for each entity of the graph, the computed entity embeddings; and
repeating, for unseen entities, steps (a) and (b) and using the trained ML model to perform inductive predictions for the unseen entities.

15. A tangible, non-transitory computer-readable medium having instructions thereon which, upon being executed by one or more processors, alone or in combination, provide for execution of a method for inductive learning on graphs, wherein a graph includes a plurality of entities, wherein relationships exist between the plurality of entities, and wherein the plurality of entities and relationships have a name string, the method comprising:
(a) creating for each entity of the graph a related text corpus, based on a respective name string of each entity;
(b) using a pretrained language model to compute, from the related text corpus of each entity, a respective contextual entity embedding for each entity of the graph;
training a graph-based machine-learning, ML, model by using, for each entity of the graph, the computed entity embeddings; and
repeating, for unseen entities, steps (a) and (b) and using the trained ML model to perform inductive predictions for the unseen entities.
